(19) United States
(12) Patent Application Publication
(10) Pub. No.: US 2014/0065192 A1
(43) Pub. Date: Mar. 6, 2014

(54) SOUR GELLED EMULSIONS

(71) Applicant: Andrzej Siwek, Tromso (NO)

(72) Inventor: Andrzej Siwek, Tromso (NO)

(73) Assignee: AYANDA GROUP AS, Tromso (NO)

(21) Appl. No.: 13/877,599

(22) PCT Filed: Oct. 6, 2011
(86) PCT No.: PCT/GB11/51915

§ 371 Date: Nov. 13, 2013

(30) Foreign Application Priority Data
Oct. 6, 2010 (GB) 1016900.1

(51) Int. Cl. A61K 47/12; A61K 9/06

(52) U.S. Cl. CPC A61K 47/12; A61K 9/06
USPC 424/400; 514/549

(57) ABSTRACT

The invention relates to an orally administrable, sour-tasting chewable composition in unit dosage form comprising an oil-in-water emulsion in which the aqueous phase is gelled and comprises at least one physiologically acceptable acid and at least one buffering agent and in which the oil phase comprises a physiologically tolerable unsaturated fatty acid ester.

This invention relates to oral pharmaceutical and nutraceutical compositions in the form of sour-tasting, chewable emulsions comprising a soft, gelled oil-in-water emulsion containing physiologically tolerable unsaturated fatty acid ester oils, and to methods of treatment of a human subject therewith.

Many unsaturated fatty acid ester oils have an unpleasant taste and/or odour. Fish oils in particular are often unpleasant to ingest, due to foul taste, mouthfeel or smell. Accordingly, any therapeutic or prophylactic dosage regime which involves the consumption of unpleasant tasting dose units or nasty smelling dose units is inherently at risk of patient non-compliance, especially when the patient is a child or is elderly.

As anyone who, in childhood, has been on the receiving end of fish oils will recall, the taste, mouthfeel and smell can be vile. In part this is due to the sensitivity to oxidation of the fish oil. As a result fatty acid ester oils tend to be administered in capsule form, containing liquid oil within a soft gel casing. Such capsule casings are usually made from mammalian gelatin, typically of porcine or bovine origin. In order to deliver a reasonable dose of the oil, the capsules tend to be rather large, sufficiently large indeed to cause problems swallowing them for the young and the elderly. As a result, ingestion often involves the capsule being chewed and bursting in the mouth releasing the unpleasantly tasting oil contents.

Many nutraceutical and drug substances are formulated as suspensions. Such suspensions often have an unpleasant taste and/or odour, are cumbersome and have limited stability. In some cases suspensions require the addition of clean water before consumption. Such suspensions are preferably refrigerated after dilution with water in order to avoid the decrease in stability of the diluted suspension, especially in hot climates.

There is thus a continuing need for improved oral administration forms for unsaturated fatty acid ester oils and for unsaturated fatty ester oil-containing pharmaceutical compositions which may be administered orally with increased ease of oral intake and better patient compliance.

In WO 2007/085840 we reported chewable set oil-in-water gelled emulsions of unsaturated fatty esters which go a long way in masking the taste and odour of the fatty ester emulsified therein. There is, however, a need to more thoroughly disguise the taste and odour of the unpleasant tasting oil component and/or drug substance in order to improve patient compliance.

We have now surprisingly found that bad odour and taste of unsaturated fatty acid esters can be further masked without destroying the gelled nature of the chewable emulsion by inclusion of at least one physiologically acceptable acid or salt thereof in the aqueous phase.

Contrary to prior art solutions, the compositions of the present invention do not merely include a flavouring to mask the taste of the unsaturated fatty acid esters, instead we have surprisingly found that the sourness of the compositions of the invention tricks the mouth into not tasting the unsaturated fatty acid esters. For example, the usual reaction to compositions comprising fish oil does not result from consumption of the compositions of the invention as the taste and smell of the fish oil is covered up by the tongue's reaction to sourness.

Thus viewed from one aspect the invention provides an orally administrable, sour-tasting, chewable composition in unit dosage form comprising an oil-in-water emulsion in which the aqueous phase is gelled and comprises at least one physiologically acceptable acid or salt thereof and at least one buffering agent and in which the oil phase comprises a physiologically tolerable unsaturated fatty acid ester.

Viewed from a further aspect, the invention provides an orally administrable, sour-tasting, chewable composition in unit dosage form comprising a gelled double emulsion, preferably a water-in-oil-in-water double emulsion, comprising at least one physiologically acceptable acid or salt thereof and at least one buffering agent in an aqueous phase and a physiologically tolerable unsaturated fatty acid ester in an oil phase.

The term unsaturated fatty acid ester oil is used herein to relate to acyl glycerides and phospholipids, i.e. compounds comprising an unsaturated fatty acid side chain linked by an ester group to an “alcohol” (e.g. polyol) residue. Such compounds are important dietary sources of fatty acids, in particular polyunsaturated fatty acids (PUFAs) and more especially the essential fatty acids. They may also serve as sources for dietary replacements of essential fatty acids, e.g. of conjugated linoleic acid (CLA) which may be used in a weight reduction diet. Particularly important essential fatty acids include the ω-3, ω-6 and ω-9 acids such as eicosapentaenoic acid (EPA), docosahexaenoic acid (DHA). Oleic acid is an important ω-9 acid. Other fatty acids commonly used in nutraceuticals and pharmaceuticals include arachidonic acid (AA), alpha linolenic acid (ALA), conjugated linolenic acid (CLN), dihomo-gamma linolenic acid (DGLA) and gamma linolenic acid (GLA). Such fatty acids typically will contain 12 to 26 carbons, more typically 16 to 22 carbons, and will have a saturated or mono- or poly-ethylenically-unsaturated hydrocarbyl chain.

Typical dietary sources of such fatty acid ester oils include lipids such as animal, fish, plant or microorganism triglycerides and phospholipids, especially the triglycerides. Mono or diglycerides however can equally be used as can other esters, e.g. lower alkyl (e.g. C₁₋₆ alkyl, for example ethyl) esters as well as free fatty acids or physiologically acceptable salts thereof and fatty acid ester waxes. Particularly important sources are fish oils, in particular oily fish oils such as cod-liver oil, halibut-liver oil, etc. as these are rich in ω-3, ω-6 and ω-9 fatty acids.

The term “chewable” is used herein in its conventional meaning within the pharmaceutical and nutraceutical industry. That is the composition is in a form which can be broken or fragmented by chewing. The chewable compositions of the invention may be pharmaceuticals, but preferably are nutraceuticals.

The oil phase of the emulsion will typically comprise a physiologically tolerable unsaturated fatty acid ester oil as described above, especially an acyl glyceride or a fatty acid ethyl ester, and in particular a fish or plant triglyceride. More preferably, it contains a fish oil. Particularly preferably, the oil phase of the emulsion will further comprise olive oil. Besides such oils, or mixtures thereof, the oil phase may also if desired contain physiologically tolerable lipid soluble materials, e.g. vitamins, antioxidants, flavourings, colours and other physiologically active materials. If desired, the oil phase may be constituted in whole or part by a phospholipid, in particular a marine (e.g. pelagic fish or shellfish, for example krill) phospholipid. The oil phase preferably contains 25 to 100% of the recommended daily dosage for one or more essential fatty acids, especially EPA and/or DHA. Typically the oil phase will constitute 0.05 to 5 g, preferably 0.1 to 3 g, especially 0.2 to 2 g, particularly 0.3 to 1.25 g, more particularly 0.4 to 0.75 g, per dose unit. Alternatively put, the oil phase preferably constitutes 5 to 75% wt., especially 30 to 50% wt., e.g. 40 to 50% wt. of the dose unit.

The oil phase of the oil-in-water emulsion may also contain solubilisers in order to increase the solubility of the drug substance in the oil phase. Suitable solubilisers would be known to a person skilled in the art and include Chremophor EL™, castor oil, Tween 80™, Solutol™ HS15, Lutrol™ and Olestra.

The aqueous phase of the emulsion comprises water and a physiologically tolerable gelling agent, preferably a saccharide (e.g. an oligosaccharide or polysaccharide), a protein or a glycoprotein. Suitable gelling agents are well known in the food, pharmaceutical and nutraceutical industries and several are described for example in Phillips et al. (Ed.) “Handbook of hydrocolloids”, Woodhead Publishing, Cambridge, UK, 2000. The gelling agents are preferably materials capable of undergoing a sol-gel transformation, e.g. under the influence of a change in physiochemical parameters such as temperature, pH, presence of metal ions (e.g. group 1 or 2 metal ions), etc.

Preferred for use as the gelling agent is starch, carrageenan, pectin, agar, gelatin, gellan, or alginate (e.g. sodium alginate or a mixture of an alginate and glucono-delta-lactone (GDL)), or a mixture of two or more of these. Alternatively or additionally, gum arabicum may be used. The use of fish gelatins is especially preferred. The gelatins described in WO 2007/085835 and WO 2007/085840 may be used as gelling agents in the composition of the invention. A starch may be used as a gelling agent, preferably an amylose or amylopectin.

The aqueous phase of the emulsion further comprises at least one physiologically acceptable acid or salt thereof and at least one buffering agent, preferably wherein said acid and buffering agent are selected from those acids and buffering agents suitable for use in the food and beverage industry. The aqueous phase of the composition of the invention is thus a buffered solution.

Preferred physiologically acceptable acids for use in the composition of the invention are weak acids, particularly fruit acids.

The term weak acid is used herein in its conventional scientific meaning. That is a weak acid is an acid that dissociates incompletely. Such acids have a pKₐ in the range of 1.74 to 15.74, i.e. the acid dissociation constant Kₐ for weak acids according to the invention is in the range 1.8·10⁻¹⁶ to 55.5.

Particularly preferably, the physiologically acceptable acids for use in the composition of the invention are selected from among citric acid, malic acid, lactic acid, phosphoric acid and acetic acid, preferably, citric acid and malic acid, e.g. malic acid. The acids may be uncoated or coated with edible coatings, e.g. microencapsulated. Coated acids may be used to delay the onset of sourness and prolong the sour taste of the compositions upon chewing.

If the acid is coated, the volume component of coating material is preferably in the range of about 3 to 40%, particularly about 5 to 20%, particularly preferably 10 to 15%, e.g. 10%. Preferably, the coated acids for use in the present invention are commercially available.

Suitable acid coating materials include hardened fats such as hardened animal or vegetable fats or hardened plant oils, preferably, food grade paraffin wax, e.g. carnauba wax. Also suitable as coating materials are water soluble encapsulating substances such as modified starch or gelatin.

Preferably, the concentration of acid in the compositions of the invention is at least 0.005% wt. of the aqueous phase, particularly preferably, at least 1% wt., e.g. 2% wt. The concentration of the acid should not be so high as to result in a pH of less than 3. Typically, this means the concentration of the acid will be less than 10% wt. of the aqueous phase, preferably less than 5% wt., e.g. 4% wt.

The term buffering agent is used herein in its conventional meaning within the pharmaceutical, nutraceutical and food industries. That is the buffering agent resists change in pH of the solution of which it is an active component.

Preferred buffering agents for use in the composition of the invention are the potassium and sodium salts of acids selected from among citric acid, sorbic acid, lactic acid, acetic acid, phosphoric acid, malic acid, fumaric acid, tartaric acid and benzoic acid, preferably, sodium citrate or sodium maleate, e.g. sodium citrate, particularly trisodium citrate.

Preferably, the buffering capacity of the aqueous phase of the compositions of the invention is sufficient to maintain a pH between 3 and 7, preferably between 3.5 and 6, particularly between 4 and 5, e.g. 4.5. This results in an aqueous phase with a pH that the mouth can tolerate while still tasting sour.

The weight ratio of acid: buffer salt is preferably in the range 0.5 to 10, particularly 0.7 to 5, particularly preferably 0.8 to 3, e.g. 0.95.

Besides the gelling agent, at least one buffering agent, at least one physiologically acceptable acid or salt thereof, water, preferably purified water, and any required gelling initiator, other physiologically tolerable materials may be present in the aqueous phase, e.g. emulsifiers, emulsion stabilizers, pH modifiers, viscosity modifiers, sweeteners, fillers, vitamins (e.g. vitamin C, thiamine, riboflavin, niacin, vitamin B6, vitamin B12, folacin, panthotenic acid), minerals, aromas, flavours such as lemon flavour, colours such as beta-carotene, physiologically active agents, etc. It is especially preferred that a lipophilic antioxidant, e.g. vitamin E, be included in the oil phase. Other vitamins which may be present in the oil phase are vitamin A, vitamin D and vitamin K. Alternatively or additionally, tocopherols may be present, e.g. D-α-tocopherol. Such further components are used widely in the food, pharmaceutical and nutraceutical industries. The use of cellulose derivatives (e.g. hydroxy methyl propyl cellulose) as emulsion stabilizers is especially preferred.

The pH of the aqueous phase of the emulsion is preferably in the range 2 to 9, particularly 3 to 7.5.

The aqueous phase preferably has a gelling temperature in the range 10 to 30° C., more preferably 15 to 28° C., and a melting temperature in the range 20 to 80° C., more preferably 24 to 60° C., especially 28 to 50° C.

Where a sweetener is included in the aqueous phase, this will typically be selected from natural sweeteners such as sucrose, fructose, glucose, reduced glucose, maltose, xylitol, maltitol, sorbitol, mannitol, lactitol, isomalt, erythritol, polyglycitol, polyglucitol and glycerol and artificial sweeteners such as aspartame, acesulfame-K, neotame, saccharine, sucralose. The use of non-cariogenic sweeteners is preferred.

Examples of drug substances that may be included in the capsules of the invention include for example analgesics (e.g. paracetamol and acetyl salicylic acid), antibiotics (e.g. penicillin), antihelminthics (e.g. abamectin), antifungals (e.g. imidazole antifungals such as miconazole) and antihistamines (e.g. loratadine). Many other drug substances may be used, e.g. anti-cancer agents, cardiovascular agents, mood-altering drugs, etc.

Preferably the overall dose unit weight of the compositions of the invention will be 0.25 to 3 g, especially 0.5 to 2.5 g, more especially 0.75 to 2 g.

Viewed from a further aspect the invention provides a method of treatment of a human by oral administration of an effective amount of a drug substance in oil form or dissolved in an oil, the improvement comprising administering said active agent in a sour-tasting, chewable emulsion according to the invention. The method may thus typically be a method of treatment of a disease or ailment (e.g. pain), a method of nutritional supplementation (e.g. with a triglyceride) or a method of reducing weight.

Emulsion formation may be effected by conventional techniques; however emulsification under a non-oxidising gas, e.g. nitrogen, is preferred. Likewise, the components of the emulsion are preferably degassed before emulsification and handling and packaging of the set emulsion is preferably performed under such a gas.

The dose units of the emulsion may be formed for example by moulding, extrusion or cutting or the like. For adult use, the dose units are preferably in tablet or lozenge form; however for child use they may conveniently be presented in child-friendly form, e.g. geometric shapes such as rods, strips and tubes, or animal, doll, or vehicle shapes, for example the shape of a popular cartoon character.

Dose units may be coated if desired, e.g. as described in WO 2007/085835. The use of gelatin coatings is preferred.

If desired, the compositions may be double emulsions, e.g. water-in-oil-in-water emulsions or oil-in-water emulsions containing two different oil phases. These may be prepared conventionally, e.g. by mixing two oil-in-water emulsions before gelling begins or by emulsifying a water-in-oil emulsion into a gelling agent containing aqueous phase.

The gelled emulsions of and used according to the invention may be produced as described in WO 2007/085835, WO 2007/085840, WO 2010/041015 and WO 2010/041017, the contents of which are hereby incorporated by reference.

It is particularly preferred that the compositions according to the invention contain a citrus flavour (e.g. orange or lemon oil) in order to mask any remaining oil taste on chewing. It is also particularly preferred that the compositions according to the invention contain xylitol, e.g. as 0.5 to 50% wt., preferably 1 to 40% wt., e.g. 15 to 40% wt., in order to mask both taste and mouth feel. These may be in the aqueous phase or the oil phase (e.g. as a water-in-oil-in water double emulsion), or both; however inclusion in the aqueous phase will generally be sufficient.

The dose units of the compositions of the invention are preferably individually packaged in air-tight containers, e.g. a sealed wrapper or more preferably a blister of a blister pack. Viewed from a further aspect the invention provides a package comprising an air-tight compartment containing one dose unit of a composition according to the invention.

The packages according to the invention are preferably in the form of blister packs containing at least two dose units, e.g. 2 to 100, preferably 6 to 30. A blister pack, as is well known, usually comprises a plastic sheet base having moulded indentations or trays in which the item to be packed is placed. The pack is normally sealed with a foil, generally metal or a metal/plastic laminate, generally by heating the areas between the indentations or trays.

The packages according to the invention are preferably filled under a non-oxidising gas atmosphere (e.g. nitrogen) or are flushed with such a gas before sealing.

Of course, in place of unsaturated fatty acid esters, saturated fatty acids and their esters could also be used and this forms a further aspect of the invention.

The invention will now be described further with reference to the following non-limiting Examples.

### EXAMPLE 1

### Sour Tasting Chewable Emulsion

Aqueous phase:

| | |
|---|---|
| Gelatin | 6.3% wt. |
| Gum arabicum | 1.5% wt. |
| Sorbitol | 9.5% wt. |
| Xylitol | 22.3% wt. |
| Coated malic acid 90% | 1.4% wt. |
| Trisodium citrate bihydrate | 1.2% wt. |
| Flavour | 1.2% wt. |
| Sucralose | 0.01% wt. |
| Colouring | 0.1% wt. |
| Water | ad 60% wt. |

Marine oil (e.g. commercially available fish liver oil) 40% wt.

The gelatin is added to the water and allowed to swell for 30 min. The gelatin solution is then heated to 70° C. under continuous stirring for 45 min. The xylitol and sorbitol are then added to the solution and allowed to dissolve under stirring for 30-60 min. The coated malic acid, trisodium citrate bihydrate, flavour, sucralose and colour are then added while stirring. The solution is mixed for 30 min before stirring is stopped and the solution is left for 30 min.

The marine oil and the aqueous solution are emulsified in a weight ratio of 1:2 at 45-50° C. using an ultra turrax. The resultant solution is degassed under vacuum to remove air bubbles. When the emulsion is smooth, soft cores are produced by moulding and left to gel for 30 min at 22 C. The cores are dried to reduce the content of water to approximately 10% wt.

### EXAMPLE 2

### Sour Tasting Chewable Emulsion

Aqueous phase:

| | |
|---|---|
| Gelatin: | 9.4% wt. |
| Gum arabicum: | 1.3% wt. |
| Sorbitol: | 13.5% wt. |
| Xylitol: | 31.7% wt. |
| DL- malic acid, uncoated | 2.6% wt. |
| Trisodium citrate bihydrate | 2.4% wt. |
| Flavour: | 1.1% wt. |
| Sweetener: | 0.05% wt. |
| Colouring: | 0.31% wt. |
| Water: | ad 80% wt. |
| Omega-3 oil concentrate | 20% wt. |

Sour-tasting chewable emulsions are prepared analogously to Example 1.

### EXAMPLE 3

### Sour Tasting Chewable Emulsion

Aqueous phase:

| | |
|---|---|
| Gelatin: | 5.7% wt. |
| Gum arabicum: | 3.0% wt. |
| Sorbitol: | 9.3% wt. |
| Xylitol: | 22% wt. |
| DL- malic acid, uncoated | 1.8% wt. |
| Sodium citrate | 3.6% wt. |
| Flavour: | 0.9% wt. |
| Colouring: | 0.01% wt. |
| Water: | ad 63.3% wt. |
| Fish oil concentrate | 36.7% wt. |

Sour-tasting chewable emulsions are prepared analogously to Example 1.

### EXAMPLE 4

### Sour Tasting Chewable Emulsion

Aqueous phase:

| | |
|---|---|
| Gelatin: | 7.2% wt. |
| Gum arabicum: | 1.5% wt. |
| Sorbitol: | 15.2% wt. |
| Xylitol: | 29.3% wt. |
| DL- malic acid, uncoated | 1.8% wt. |
| Sodium citrate | 3.6% wt. |
| Flavour: | 1.0% wt. |
| Colouring: | 0.01% wt. |
| Water: | ad 76.6% wt. |
| Fish oil concentrates | 19.5% wt. |
| Olive oil | 3.3% wt. |
| Vitamin E | 0.55% wt. |

Sour-tasting chewable emulsions are prepared analogously to Example 1.

### EXAMPLE 5

### Blister Packs

The emulsion cores of Examples 1, 2, 3 and 4 are filled into plastic blister pack trays over which a plastic/metal foil laminate is heat sealed.

### EXAMPLE 6

### Chewable Strips

Before setting, the emulsions of Examples 1, 2, 3 and 4 are extruded into strips which are then sealed into individual plastic/metal foil laminate envelopes.

1. An orally administrable, sour-tasting chewable composition in unit dosage form comprising an oil-in-water emulsion in which the aqueous phase is gelled and comprises at least one physiologically acceptable acid and at least one buffering agent and in which the oil phase comprises a physiologically tolerable unsaturated fatty acid ester.

2. A composition as claimed in claim 1 wherein the acid is a weak organic, preferably a fruit acid.

3. A composition as claimed in claim 2 wherein the acid is selected from citric acid, malic acid, lactic acid, phosphoric acid and acetic acid, preferably, citric acid and malic acid.

4. A composition as claimed in claim 1 wherein the buffering agent is a potassium or sodium salt of an acid selected from among citric acid, sorbic acid, lactic acid, acetic acid, phosphoric acid, malic acid, fumaric acid, tartaric acid and benzoic acid, preferably, sodium citrate or sodium maleate.

5. A composition as claimed in claim 1 wherein the weight ratio of acid: buffer is in the range 0.5 to 10, preferably in the range 0.7 to 5.

6. A composition as claimed in claim 1 wherein the pH of the composition is in the range 2 to 9.

7. A composition as claimed in claim 1 wherein the fatty acid ester is a physiologically tolerable triglyceride, preferably a fish oil.

8. A composition as claimed in claim 1 wherein said aqueous phase comprises a gelling agent selected from gelatin, polysaccharides, and mixtures thereof.

9. A composition as claimed in claim 1 wherein said aqueous phase comprises gelatin and carrageenan.

10. A composition as claimed in claim 1 containing at least 1% wt. xylitol.

11. A composition as claimed in claim 1 containing a citrus flavour.

12. A composition as claimed in claim 1 wherein said aqueous phase comprises a water soluble vitamin.

13. A composition as claimed in claim 1 in animal, doll, vehicle, rod, strip or tube shape.

14. A package comprising an air-tight compartment containing one dose unit of a composition as claimed in claim 1.

15. A package as claimed in claim 14 in the form of a blister pack.

16. A method of treatment of a human by oral administration of an effective amount of an active agent in oil form or dissolved in an oil, the improvement comprising administering said active agent in a chewable composition according to claim 1.

17. A method as claimed in claim 16 wherein said active agent is an analgesic.

18. A method as claimed in claim 16 wherein said active agent is a triglyceride.
